(19) United States
(12) Patent Application Publication
(10) Pub. No.: US 2025/0108362 A1
(43) Pub. Date: Apr. 3, 2025

(54) NANOMATERIAL-BASED ADSORPTION PROCESSING AND CATALYSIS

(71) Applicant: DREXEL UNIVERSITY, Philadelphia, PA (US)

(72) Inventors: Michel W. Barsoum, Moorestown, NJ (US); Hussein O. Badr, Philadelphia, PA (US); Joshua Snyder, Sparks, MD (US)

(21) Appl. No.: 18/978,462

(22) Filed: Dec. 12, 2024

Related U.S. Application Data
(63) Continuation of application No. 18/832,624, filed on Jul. 24, 2024.
(60) Provisional application No. 63/309,391, filed on Feb. 11, 2022.

(51) Int. Cl. B01J 23/755; B01J 35/39; B01J 35/58; C02F 1/30; C02F 1/467; C02F 1/72; C02F 101/38

(52) U.S. Cl. CPC B01J 23/755; B01J 35/39; B01J 35/58; C02F 1/30; C02F 1/4672; C02F 1/725; C02F 2101/38; C02F 2305/08; C02F 2305/10

(57) ABSTRACT

A method, comprising: contacting a composition comprising metal oxide-based nanofilaments to an initial sample that comprises a metal ion and/or a metal under such conditions that at least some of the metal ion and/or the metal associates with the metal oxide nanofilaments, the metal oxide nanofilaments having a lepidocrocite structure and the metal oxide nanofilaments optionally comprising titanium, and optionally comprising illuminating the composition and metal oxide nanofilaments and/or metal.

### CROSS-REFERENCE TO RELATED MATTERS

The present application is a Continuation of U.S. patent application Ser. No. 18/832,624, “Nanomaterial-Based Adsorption Processing And Catalysis” (filed Jul. 24, 2024); which is the National Stage Application of International Patent Application No. PCT/US2023/062394, “Nanomaterial-Based Adsorption Processing And Catalysis” (filed Feb. 10, 2023); which claims priority to and the benefit of U.S. patent application No. 63/309,391, “Nanomaterial-Based Adsorption Processing And Catalysis” (filed Feb. 11, 2022). All foregoing applications are incorporated herein by reference in their entireties for any and all purposes.

### TECHNICAL FIELD

The present disclosure relates to the field of two-dimensional materials.

### BACKGROUND

Removing certain undesired species (e.g., urea) from samples is an important process with applications in industry, healthcare, and the environment. Effecting such adsorption in a consistent and effective manner can, however, be challenging. Accordingly, there is a need for materials capable of adsorbing unwanted species from samples, solutions and suspensions and also for methods of effecting such adsorption.

### SUMMARY

In meeting the described needs, the present disclosure provides a method, comprising: to an initial sample that has an initial concentration of urea in solution, contacting a composition comprising a metal oxide nanofilaments under such conditions that at least some of the urea adsorbs to the metal oxide nanofilaments so as to give rise to a final concentration of urea in the solution, the metal oxide nanofilaments optionally comprising titanium, the metal oxide nanofilaments optionally comprising carbon, the structure of the oxide nanofilaments optionally being an anatase structure or a lepidocrocite structure, and further optionally comprising illuminating the composition and the urea. An oxide nanofilament can be a metal oxide nanofilament having a one-dimensional lepidocrocite (1DL) structure. Such methods can be used in, e.g., dialysis applications, such as for patients who suffer from kidney ailments.

Also provided is a device for removing urea from an initial aqueous solution of urea, the device comprising an exchangeable cartridge of metal oxide nanofilaments composition through which the initial aqueous solution is directed to pass, the passage adapted to allow the initial aqueous urea solution to contact the metal oxide nanofilaments contained in the cartridge, the metal oxide nanofilaments optionally comprising titanium, the metal oxide nanofilaments optionally comprising carbon, and the structure of the oxide nanofilaments optionally being an anatase structure or a lepidocrocite structure. Such devices can be used in, e.g., dialysis applications.

Also provided is a method, comprising: contacting a composition comprising metal oxide-based nanofilaments to an initial sample that comprises a metal ion and/or a metal under such conditions that at least some of the metal ion and/or the metal associates with the metal oxide nanofilaments, the metal oxide nanofilaments optionally comprising titanium and the metal oxide nanofilaments optionally having an anatase structure or a lepidocrocite structure; and optionally comprising illuminating the composition and metal oxide nanofilaments and/or metal.

Further provided is a composition, comprising a metal oxide nanofilaments having at least two salts associated therewith.

Additionally provided is a method, comprising: contacting (i) a composition comprising metal oxide nanofilaments with at least one metal of a metal salt associated therewith and (ii) solution to as to catalyze a reaction within the solution, the metal oxide nanofilaments optionally comprising titanium, the metal oxide nanofilaments optionally having an anatase structure or a lepidocrocite structure. The metal of the metal salt can be different from the metal of the metal oxide nanofilaments.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIG. 1 provides oxygen evolution reaction polarization curves in 0.1 M KOH for TCO (grey), TCO+Ni (green), TCO+Ni/Fe (red), and commercial IrO₂ catalyst (black).

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. Such description, however, should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant FIGURES and numerical values which differ from the stated value by less than the experimental error of conventional measurement technique of the type described in the present application to determine the value.

All ranges disclosed herein are inclusive of the recited endpoint and independently of the endpoints (e.g., “between 2 grams and 10 grams, and all the intermediate values includes 2 grams, 10 grams, and all intermediate values”). The endpoints of the ranges and any values disclosed herein are not limited to the precise range or value; they are sufficiently imprecise to include values approximating these ranges and/or values. All ranges are combinable.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. In at least some instances, the approximating language may correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4. Further, the term “comprising” should be understood as having its open-ended meaning of “including,” but the term also includes the closed meaning of the term “consisting.” For example, a composition that comprises components A and B may be a composition that includes A, B, and other components, but may also be a composition made of A and B only. Any documents cited herein are incorporated by reference in their entireties for any and all purposes.

### EXAMPLE

The following examples are illustrative only and does not serve to limit the scope of the present disclosure or the appended claims.

Provide herein is an example methodology for improving the catalytic capabilities of a TCO material through incorporation of a variety of metallic species through an exchange process.

Without being bound to any particular theory, the exchange process can include convective mixing of the colloidal TCO material in an aqueous solution containing one or more inorganic/organic salts of transition metal species. Convective stirring without the addition of external driving force, e.g., temperature, potential, reactive agent, reducing agent, etc., yields a self-limiting incorporation of transition metals into the TCO material. This occurs both with and without exchange of Ti atoms in the parent material. Analysis of the composition of the material demonstrates incorporation of the new transition metal, indicating that it is not simply intercalated as an ion in between the layers. The electrochemical redox behavior can be indicative of the new transition metal being a reduced species. This exchange process does not occur on other oxide materials, indicating an aspect of the TCO that favors this exchange and incorporation of reduced metallic species into the material. This process is suitable for a variety of metals, including transition metals whose equilibrium potentials are higher than titanium. This process has been demonstrated to be self-limiting as the quantity of metal exchange and overall performance of material does not change with increased duration of the exchange process.

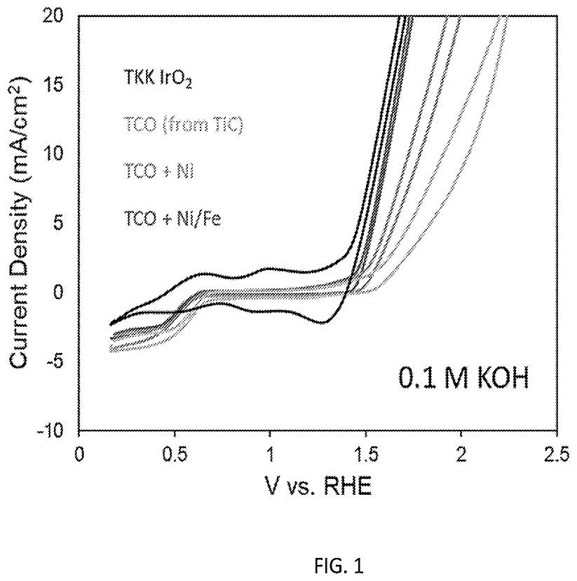

Following TCO synthesis, Ni and Ni/Fe are incorporated into the TCO material. FIG. 1 demonstrates the impact of Ni and Ni/Fe additives to the TCO by measuring the activity of the material for the oxygen evolution reaction (OER). Following incorporation of Ni, a marked increase in the activity of the material, as measured by an increased current at a given potential above 1.23 V versus the reversible hydrogen electrode (RHE), over the plain TCO is observed. Incorporation of both Ni and Fe into the TCO using the described exchange process results in a further increase in OER performance. Plotted for comparison is the current industrial state of the art OER catalyst, IrO₂. The Ni/Fe incorporated TCO yields an OER performance that matches that of the current industrial state of the art. This result demonstrates the utility of the described approach in tuning the catalytic capabilities of the TCO material.

### Aspects

The following Aspects are illustrative only and do not limit the scope of the present disclosure or the appended claims. Any part or parts of any one or more Aspects can be combined with any part or parts of any one or more other Aspects.

Aspect 1. A method, comprising: to an initial sample that has an initial concentration of urea in solution, contacting a composition comprising metal oxide nanofilaments under such conditions that at least some of the urea adsorbs to the metal oxide nanofilaments so as to give rise to a final concentration of urea in the solution, the metal oxide nanofilaments optionally comprising titanium, the metal oxide nanofilaments optionally comprising carbon, the structure of the oxide nanofilaments optionally being an anatase structure or a lepidocrocite structure, and further optionally comprising illuminating the composition and the urea.

Titanium oxide nanofilaments are considered especially suitable. Example of one dimensional nanofilaments are described in Badr, et al., “Bottom-Up, Scalable Synthesis Of Anatase Nanofilament-Based Two-Dimensional Titanium Carbo-Oxide Flakes,” Materials Today 2021 (https://doi.org/10.1016/j.mattod.2021.10.033) and Badr, et al., “On the structure of one-dimensional TiO₂ lepidocrocite”, Matter 2023 (https://doi.org/10.1016/j.matt.2022.10.015). It should be understood that the nanofilaments can be comprised in the form of mesoporous materials (e.g., powders), in the form of sheets, and other forms. It should be understood that nanofilaments can be tubular in nature.

It should be understood that the nanofilaments can be comprised in the form of mesoporous materials (e.g., powders), in the form of sheets, and other forms.

Aspect 2. The method of claim 1, wherein the initial concentration of urea is in a range of from 10 mmol/L to 1000 mmol/L, or is initially in a concentration range from 15 to 40 mg/dL mg/dL, and the final concentration is at least 10% less than the initial concentration, and wherein the sample solution is or comprises blood or a blood product and conditions do not compromise the utility of the blood or blood product for later use by a human patient.

Aspect 3. The method of claim 2, wherein the final concentration is at least 30% less than the initial concentration.

Aspect 4. The method of claim 3, wherein the final concentration is at least 50% less than the initial concentration.

Aspect 5. The method of claim 4, wherein the final concentration is at least 70% less than the initial concentration.

Aspect 6. A device for removing urea from an initial aqueous solution of urea, the device comprising an exchangeable cartridge of metal oxide nanofilaments composition through which the initial aqueous solution is directed to pass, the cartridge adapted to allow the initial aqueous urea solution to contact the metal oxide nanofilaments contained in the cartridge, the metal oxide nanofilaments optionally comprising titanium, the metal oxide nanofilaments optionally comprising carbon, and the structure of the oxide nanofilaments optionally being an anatase structure or a lepidocrocite structure.

Aspect 7. The device of claim 6, wherein the device is adapted to allow the initial aqueous solution of urea to percolate through at least a portion of the metal oxide nanofilaments.

Aspect 8. The device of claim 6, wherein the cartridge comprises channels coated with the metal oxide nanofilaments.

Aspect 9. The device of claim 6, wherein the cartridge comprises channels coated with the metal oxide nanofilaments, the metal oxide nanofilaments comprising titanium and the metal oxide nanofilaments having an anatase structure or a lepidocrocite structure.

Aspect 10. The device of claim 6, wherein the metal oxide nanofilaments composition is present as a plurality of stacked layers.

Aspect 11. A method, comprising: contacting a composition comprising metal oxide-based nanofilaments to an initial sample that comprises a metal ion and/or a metal under such conditions that at least some of the metal ion and/or the metal associates with the metal oxide nanofilaments, the metal oxide nanofilaments optionally comprising titanium and the metal oxide nanofilaments optionally having an anatase structure or a lepidocrocite structure; and optionally comprising illuminating the composition and metal oxide nanofilaments and/or metal.

Aspect 12. The method of claim 11, wherein the metal ion and/or metal comprises As, Pb, Cd, Cr, Ni, Zn, Co, or Mn, Ir, Au, Ru, Re, Pt, Pd, Ag or any ion thereof.

Aspect 13. The method of claim 11, wherein the illuminating comprises xenon lamp illumination.

Aspect 14. The method of claim 11, wherein the illuminating comprises natural light

Aspect 15. The method of any one of claims 11-14, wherein the association (which can be adsorption) is essentially complete within about 1 hour.

Aspect 16. The method of claim 15, wherein the adsorption is essentially complete within about 15 minutes.

Aspect 17. The method of claim 16, wherein the adsorption is essentially complete within from about 2 to about 3 minutes.

Aspect 18. A composition, comprising a metal oxide nanofilaments having at least two different metal salts associated therewith. The metals of the at least two salts can be different than the metal of the metal oxide nanofilaments.

Aspect 19. The composition of claim 18, wherein at least one of the at least two different metal salts comprises nickel.

Aspect 20. The composition of claim 18, wherein at least one of the at least two different metal salts comprises iron.

Aspect 21. A method, comprising: contacting (i) a composition comprising metal oxide nanofilaments with at least one metal of a metal salt associated therewith and (ii) solution to as to catalyze a reaction within the solution, the metal oxide nanofilaments optionally comprising titanium, the metal oxide nanofilaments optionally having an anatase structure or a lepidocrocite structure. The metal of the metal salt can be different from the metal of the metal oxide nanofilaments.

Aspect 22. The method of claim 21, wherein the reaction proceeds more rapidly than if the metal oxide nanofilaments lacked the at least one salt associated therewith.

Aspect 23. The method of any one of claims 21-22, wherein the at least one salt comprises iron, nickel, or any combination thereof.

Aspect 24. The method of any one of claims 21-23, wherein the solution comprises water.

Aspect 25. The method of any one of claims 21-24, wherein the reaction is an electrochemical reaction that includes application of a current to the solution.

What is claimed:

1. A method, comprising:
contacting a composition comprising metal oxide-based nanofilaments to an initial sample that comprises a metal ion and/or a metal under such conditions that at least some of the metal ion and/or the metal associates with the metal oxide nanofilaments,
the metal oxide nanofilaments having a lepidocrocite structure and the metal oxide nanofilaments optionally comprising titanium; and
optionally comprising illuminating the composition and metal oxide nanofilaments and/or metal.

2. The method of claim 1, wherein the metal ion and/or metal comprises As, Pb, Cd, Cr, Ni, Zn, Co, or Mn, Ir, Au, Ru, Re, Pt, Pd, Ag or any ion thereof.

3. The method of claim 1, wherein the illuminating comprises xenon lamp illumination.

4. The method of claim 1, wherein the illuminating comprises natural light.

5. The method of claim 1, wherein the association is essentially complete within about 1 hour.

6. The method of claim 5, wherein the association is essentially complete within about 15 minutes.

7. The method of claim 6, wherein the association is essentially complete within from about 2 to about 3 minutes.

8. A composition, comprising a metal oxide nanofilaments having at least two different metal salts associated therewith.

9. The composition of claim 8, wherein at least one of the at least two different metal salts comprises nickel.

10. The composition of claim 8, wherein at least one of the at least two different metal salts comprises iron.

11. A method, comprising:
contacting (i) a composition comprising metal oxide nanofilaments with at least one metal of a metal salt associated therewith and (ii) solution to as to catalyze a reaction within the solution,
the metal oxide nanofilaments optionally comprising titanium, and
the metal oxide nanofilaments having a lepidocrocite structure.

12. The method of claim 1, wherein the reaction proceeds more rapidly than if the metal oxide nanofilaments lacked the at least one metal salt associated therewith.

13. The method of claim 11, wherein the at least one metal salt comprises iron, nickel, or any combination thereof.

14. The method of claim 11, wherein the solution comprises water.

15. The method of claim 11, wherein the reaction is an electrochemical reaction that includes application of a current to the solution.
